(19) United States
(12) Patent Application Publication
(10) Pub. No.: US 2003/0168063 A1
(43) Pub. Date: Sep. 11, 2003

(54) Pressure face mask and nasal mask

(76) Inventors: Anthony Joseph Gambone, Laguna Niguel, CA (US); Glen Gee, Carlsbad, CA (US); Donald Lee Brisco, Lake Elsinore, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/313,526

(22) Filed: Dec. 5, 2002

Related U.S. Application Data
(60) Provisional application No. 60/362,317, filed on Mar. 8, 2002.

(51) Int. Cl. A61M 16/10; A61M 15/00

(52) U.S. Cl. 128/203.16

(57) ABSTRACT

Disclosed is a positive pressure full-face mask comprising a foam cuff, preferably made from a non-reticulated ester polyurethane. The disclosed face mask provides a superior seal to the user's face compared with face masks with air-filled cushion or silicone gasket cuffs, while providing a more comfortable user experience. Also disclosed is a nasal mask with a foam cushion that is more comfortable to the user.

### RELATED APPLICATION

This application claims priority to U.S. Provisional Patent Application Serial No. 60/362,317, filed Mar. 8, 2002, the disclosure of which is incorporated by reference in its entirety.

### BACKGROUND OF THE INVENTION

1. Technical Field of the Invention

The present application relates generally to face masks and, more particularly, to positive pressure full-face masks and nasal masks with improved sealing and reduced irritation to the face of the user.

2. Description of the Related Art

Positive pressure full-face masks are used to provide a breathable gas above ambient pressure to a user. A positive pressure full-face mask forms a seal around the nose and mouth of a user's face, providing a leak-free interface between the gas source and the user's respiratory system. Positive pressure full-face masks are used in, for example, non-invasive positive pressure ventilators (NPPV), bag-valve-mask resuscitators (BVMR), anesthesia breathing circuits, mouth-to-mask resuscitation devices, and transport ventilators. Positive pressure full-face masks are also used in other applications, for example, in breathing apparatus used by fire fighters, aircraft pilots, miners, and the like. Full face masks are also used as industrial safety and bacterial/viral filtration masks.

A full-face mask comprises a dome and a cuff. The dome fits over the user's nose and mouth, and provides a connecting means to the source of breathable gas. The cuff, or seal, which is secured to the perimeter of the dome, provides a seal between the user's face and the dome. Ideally, the seal is gas-tight under the pressures normally used. Examples of typical cuffs in positive pressure full-face masks include air-filled cushions and silicone gaskets.

An air-filled cushion is a gas-filled, expandable tube. An air-filled cushion allows a user to adjust pressure in the cushion to optimize the seal to the user's face. When a full-face mask equipped with an air-filled cushion cuff is placed over the nose and mouth of a user, the air-filled cushion conforms to the user's face, forming a seal. Air-filled cushion cuffs often deflate, however, necessitating refilling the cushion through an air inflation tube with, for example, a syringe. For mask of this type, it is not unusual for 25% to 30% of the masks in a lot or shipment to be unusable out of the box because of completely or partially deflated seals.

Moreover, an air-filled cushion often will not acceptably seal to a face with wrinkles or other irregularities. In such cases, pressing the mask against the user's face to improve the seal is, in fact, counterproductive because pressing on the mask increases the pressure within the air-filled cushion. The increased pressure in the cushion increases the tension on the tube forming its surface, pulling the surface of tube out of any irregularities, thereby providing avenues for gas to escape.

A silicone gasket is a soft, silicone cuff shaped to conform to the user's face. Silicone gasket cuffs can irritate a user's skin, however, leading ultimately to skin rashes and ulcerations. Moreover, silicone gaskets often do not seal well to the user's face, especially around the bridge of the nose. The resulting air leaks into the user's eyes lead to eye irritation. The combination of skin and eye irritation reduces user tolerance and compliance with the treatment.

A third design for a facemask cuff is a perforated tubular membrane filled with a resilient filler material, for example foam. As the mask is pressed against the user's face, air is expelled from the tubular membrane through the perforations. The filler material conforms to the user's facial features, providing improved sealing to facial irregularities. Masks of this type are said to provide a seal of better than 40 cm H₂O. The improved sealing requires that the mask be strapped tightly to the user's face, however, which is often uncomfortable. The cuff in this type of mask is also very large and often intrudes on the user's eyes, and also does not seal well to the faces of bearded users or over tubing, for example, nasal cannulae. Facial irritation is also a problem with this type of mask, both in short term and in long term applications. Extended use may lead to a rash or even blistering.

Facemask cuffs have also been made from hydrogels. These cuffs are mounted to the dome just before use, often requiring adjustment to provide optimal results. Even when properly mounted, this type of cuff requires extra steps prior to use.

A second type of facemask is a nasal mask. Nasal masks provide air to the user's nose only. Nasal masks are typically used in continuous positive airway pressure (CPAP) therapy for obstructive sleep apnea (OSA). Because a nasal mask is worn overnight, every night, mask discomfort is a major factor in noncompliance with CPAP therapy. For example, skin irritation at the interface between the mask and the face is common. Another frequent complaint is air leaking into the user's eyes. The interface between the mask and the skin of the user in a nasal mask is typically silicone or a gel-filled silicone.

Air leakage, especially into the eyes, and skin irritation are problems for both full-face masks and nasal masks, both of which can lead to user non-compliance and dissatisfaction. Accordingly, improved face mask designs are needed to overcome these problems.

### SUMMARY OF THE INVENTION

A first embodiment of the present invention provides a positive pressure full-face mask comprising dome secured to a foam cuff, wherein the foam cuff contacts a user's face. Preferably, the cuff is made from non-reticulated polyurethane foam, more preferably, a non-reticulated, ether-type polyurethane foam,

A second embodiment provides a positive pressure full-face mask comprising a dome secured to a cuff, wherein the mask will hold about 60 cm H₂O or greater gas pressure when held against a user's face with normal hand pressure.

A third embodiment provides a method of providing a breathable gas to a user comprising placing a positive pressure full-face mask comprising a dome secured to a foam cuff, wherein the cuff contacts the user's face, over the nose and mouth of the user, applying sufficient pressure to form a seal between the facemask and the user's face, and providing a breathable gas through an inlet port on the facemask.

A fourth embodiment provides a nasal mask comprising a foam cushion. Preferably, the foam is a viscoelastic foam or a polyurethane foam.

A fifth embodiment provides a method of providing CPAP therapy to a user comprising positioning a nasal mask comprising a foam cushion over the nose of the user, applying sufficient pressure to the mask to form a seal between the nasal mask and the user's face, and providing a breathable gas through an inlet port on the nasal mask. Preferably, the foam is a viscoelastic foam or a polyurethane foam.

The disclosed full-face mask is suitable for both positive pressure and positive/negative pressure applications. Compared with an air-filled cushion full-face mask, the disclosed face mask has no air-filled cushion, and hence, no leaking, refilling, or other failure associated with air-filled cushion face masks. Preferred embodiments of the disclosed face mask also provide superior sealing to the user's face than either air-filled cushion or silicone gasket face masks, as well as improved comfort. The disclosed nasal mask is suitable for CPAP applications. The foam seal of the nasal mask provides superior sealing to the user's face, as well as improved comfort.

### BRIEF DESCRIPTION OF THE FIGURES

FIG. 1 illustrates a preferred embodiment of the disclosed full face mask.

FIG. 2 is a schematic of an apparatus used to test foam samples for gas permeability.

FIG. 3 is a graph of gas leakage for 19 non-reticulated polyurethane foam samples.

FIG. 4 is a side view of a face mask illustrating the passage of cannulae through the foam cuff.

FIG. 5 illustrates a preferred embodiment of the disclosed nasal mask.

### DETAILED DESCRIPTION OF THE PREFERRED EMBODIMENTS

A breathable gas is a gas containing sufficient oxygen to sustain a user. A breathable gas may contain inert gases, for example nitrogen, helium, or water vapor. A breathable gas may also contain anesthetics, medications, and the like in admixture. The term “user” as used herein includes persons using a full-face mask or nasal mask in both medical and non-medical applications. The terms “lpm” and “LPM” mean liters per minute. The term “ppi” means pores per inch.

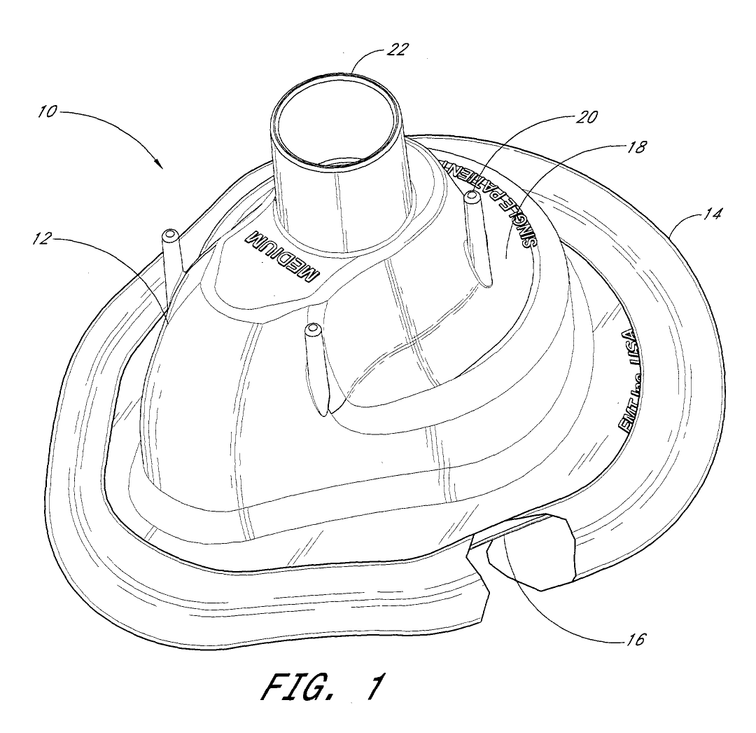

Referring to FIG. 1, a preferred embodiment of the disclosed full-face mask 10 comprises a dome 12 and a cuff 14. The cuff 14 is preferably mounted on a flange 16 on the dome 12. The dome 12 is sized to cover the nose and mouth of the user. The dome 12 is generally convex, providing clearance for the user's nose and other facial features.

In the illustrated embodiment, the cuff 14 is attached to the dome 12 at a flange 16. The cuff 14 may also contact portions of the dome 12 other than the flange 16. The flange 16 may extend outwards from the dome as shown in FIG. 1, or inwards. In the illustrated embodiment, the cuff 14 is formed with a C-shaped cross-section that engages the flange 16. In an alternative embodiment, the cuff 14 contacts the flange 16 only on the surface proximal to the user's face. The cuff 14 may be preformed and fitted on the flange 16, or alternatively, molded directly on the flange 16. In embodiments without a flange, the cuff is attached directly to the edge of the dome 12 proximal to the user's face.

The flange 16 is preferably contoured to approximate the contours of a human face, as shown in FIG. 1. The width of the flange 16 may vary along the circumference of the dome 12. For example, the flange 16 around the bridge of the nose may be narrower to avoid obstructing the user's vision. The width of the flange may also vary depending on the thickness and width of the cuff 14, as is described in greater detail below.

The dome is also preferably equipped with a ledge or indentation 18 that provides a gripping area, allowing a user to more easily position the face mask, even using only one hand. This feature is especially advantageous for users that have difficulty holding objects, for example, elderly or arthritic users. The ledge 18 may be textured to improve the user's grip. In a preferred embodiment, the ledge is approximately U-shaped, with the bottom of the “U” pointing downward when positioned on the standing or seated user's face, matching the shape of a user's hand. When the user grasps the ledge and moves the face mask towards the face, the mask is properly positioned. Consequently, a mask with this configuration may be self-administered in the dark.

The face mask 10 may be secured to the user's head with a strap or head harness that attaches to pins 20 on the dome 12. The strap is of any type known in the art for securing full-face masks, for example, a clothed neoprene spider or a head cap (not illustrated).

Breathable gas is supplied to the full-face mask through the inlet-outlet port 22. Preferably, the port is of a standard design, for example a 22 mm female conical connector according to ISO 5356-1, allowing the disclosed face mask to be integrated into the existing medical infrastructure.

The dome 12 is preferably a biocompatible material and compatible with breathable gases. The dome 12 is preferably sufficiently durable to withstand conditions of ordinary use, for example, in emergency medical operations such as bag-valve-mask resuscitation at an accident scene. Many polymeric materials are biocompatible and sufficiently strong and tough, for example, polyesters, polyamides, polycarbonates, polystyrene, acrylics, polyolefins, polyethylene, polyethylene terephthalate, silicones, and fluoropolymers. The dome may be constructed of a combination of materials. Preferred materials for the dome include polyvinyl chloride (PVC) and styrene-butadiene copolymers, for example, K-resin.

The dome 12 may also comprise reinforcing materials, for example, glass fibers, carbon fibers, polymer fibers, metal wires, or the like. The reinforcing material may also be in the form of a mesh, a band, or another structure, as would be apparent to one skilled in the art. Moreover, different parts of the dome may require additional or a different type of reinforcement, or even none at all. Preferably, the dome is transparent or translucent, or has a transparent or translucent portion or portions, allowing, for example, observing the color of a user's lips or the presence of vomitus without removing the mask.

In a preferred embodiment, the dome 12 is flexible. This flexibility allows the dome 12 to conform to the contours of a user's face, improving the seal. The thickness of the dome 12 will vary with the particular material used in its manufacture, as well as the desired flexibility. Different parts of the dome may be thicker or thinner, for example, to provide greater flexibility at the interface, or to provide rigidity, for example at the pins 20 or at the gas inlet-outlet port 22.

The portion of the cuff 14 that forms the seal to the user's face is preferably contoured to provide an acceptable seal to a variety of facial morphologies. A suitably contoured cuff provides a good seal around difficult-to-seal features including the area around the eyes, beards, elderly faces, and tubing. Contouring also reduces skin abrasion and irritation, especially around the bridge of the nose. Preferably, the portion of the cuff 14 in contact with the user's face is smooth, which would minimize irritation to the user's face. In a preferred embodiment, the portion of the cuff in contact with the user's face has a rounded cross-section. In another preferred embodiment, the cuff 14 and the flange 16 are contoured to provide clearance around the bridge of the nose, allowing the user to wear eyeglasses while wearing the mask. Suitably contouring the cuff to achieve these purposes is within the scope of the skilled artisan without excessive experimentation.

Moreover, certain areas of the face are relatively soft, for example the cheek, compared to other facial features, for example the bridge of the nose or the chin; consequently, providing additional foam in such areas compensates for the additional compliance of these facial features. The thickness of the cuff 14 also varies with the size of the mask. Preferably, the uncompressed thickness of the foam between the dome and the users face is from about 1 mm to about 50 mm, more preferably from about 2 mm to about 25 mm, most preferably from about 3 mm to about 10 mm. Typically, the cuff 14 is wide enough to cover the flange 16 of the skinward side of the mask so that the dome does not contact the user's skin under normal use. Those skilled in the art will appreciate that those areas in which the foam is thicker may be made wider to provide additional mechanical support. For example a thick but narrow piece of foam may roll or deflect sideways when compressed rather than compressing vertically. The optimal height to width ratio of the foam will vary with the type of foam and its rigidity, and is readily determined without undue experimentation. In those areas in which the foam cuff 14 is wider, the flange 16 may also be made wider to provide additional support for the cuff 14.

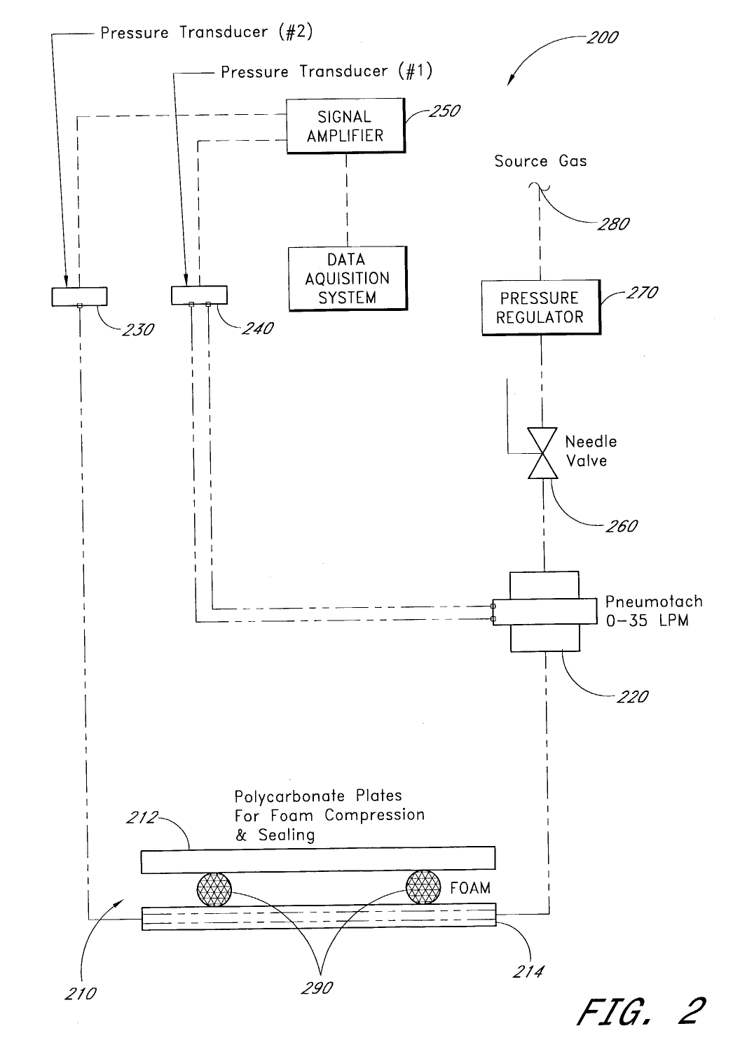

The cuff 14 is preferably made from a foam selected to provide an acceptable seal under the conditions in which positive pressure full-face masks are used. A number of foams were tested for their sealing abilities. The test apparatus is illustrated in FIG. 2. The apparatus 200 has a test fixture 210 consisting of an upper 212 and a lower 214 polycarbonate plate. The lower plate 214 has hole bored into the top that allows gas to enter the test sample and that is in fluid connection to a pneumotachometer 220 and a first pressure transducer 240. One preferred pneumotachometer 220 is a Series 4719 manufactured by Hans Rudolph, which has a flow range of about 0-160 lpm. The pneumotachometer 220 is also connected to a second pressure transducer 230. Preferred pressure transducers include a DP45-32 manufactured by Validyne with a pressure range of about 0-140 cm H₂O for the first pressure transducer 240, and DP45-14 manufactured by Validyne with a pressure range of about 0-2.25 cm H₂O for the second pressure transducer 230. The pressure transducers are connected to a data acquisition system 250, for example, a CD19A high gain carrier demodulator module manufactured by Validyne connected to a DI-720-USB 32 channel interface by DATAQ, the output of which is acquired with a personal computer. Gas enters the pneumotachometer 220 from a needle valve 260, which is downstream from a pressure regulator 270 into which a source gas 280 is fed. A preferred needle valve 260 is an SS-22RS1 valve supplied by Whitey, and a preferred pressure regulator 270 is a R74G-4AT-RM6 regulator with an outlet range of about 0150 psig manufactured by Norgren.

A sample 290 of the softest available foam of each type was placed in the test fixture 210 such that the hole in the lower plate 214 is approximately centered in the sample 290. Each piece of foam was approximately toroidal, in the shape of a face mask cuff, with an outside diameter of about 4″ and an inside diameter of about 2½″. The height of each sample 290 is provided in TABLE I. The upper plate 212 was placed on top of the sample 290. Gauge blocks (not pictured) were used to control the degree of compression on the sample 290. The source gas 280 was dry, breathable compressed air. The regulator 270 was set to 50 psig. The gas pressure at the pneumotachometer 220 was set with the needle valve 260. The gas pressure in cm H₂O required to maintain a gas flow of 5 lpm for a series of foam samples are provided in TABLE I. The pressure required to maintain a flow rate of 10 lpm for the same foam samples is provided in TABLE II. A higher gas pressure required to maintain the flow rate translates into lower gas leakage by the foam test sample.

TABLE I
Pressure Required to Maintain 5 LPM Gas Flow

| | Height, | Compression | Pressure | Compression | Pressure | Compression | Pressure |
|---|---|---|---|---|---|---|---|
| Foam^{a} | in. | (%) | (in. H₂O) | (%) | (in. H₂O) | (%) | (in. H₂O) |
| Goggle | 0.88 | 20 | 5.00 | 35 | 7.50 | 50 | 12.00 |
| Viscoelastic- | 1.00 | 25 | 0.00 | 40 | 0.00 | 50 | 0.50 |
| A | | | | | | | |
| Viscoelastic- | 1.25 | 25 | 0.00 | 40 | 0.50 | 60 | 1.50 |
| C | | | | | | | |
| Viscoelastic- | 1.00 | 25 | 1.00 | 40 | 2.50 | 50 | 3.50 |
| D | | | | | | | |
| Superseal-G | 1.00 | 25 | 0.00 | 40 | 2.00 | 50 | 4.00 |

^{a}Foams supplied by Foamex.

TABLE II
Pressure Reciuired to Maintain 10 LPM Gas Flow

| | Height, | Compression | Pressure | Compression | Pressure | Compression | Pressure |
|---|---|---|---|---|---|---|---|
| Foam^{a} | in. | (%) | (in. H₂O) | (%) | (in. H₂O) | (%) | (in. H₂O) |
| Goggle | 0.88 | 20 | 11.00 | 35 | 18.50 | 50 | 30.00 |
| Viscoelastic- | 1.00 | 25 | 0.00 | 40 | 0.00 | 50 | 1.00 |
| A | | | | | | | |
| Viscoelastic- | 1.25 | 25 | 0.5 | 40 | 0.50 | 60 | 3.50 |
| C | | | | | | | |
| Viscoelastic- | 1.00 | 25 | 3.00 | 40 | 5.00 | 50 | 8.00 |
| D | | | | | | | |
| Superseal-G | 1.00 | 25 | 1.00 | 40 | 4.00 | 50 | 8.00 |

^{a}Foams supplied by Foamex.

As shown in TABLE I and TABLE II, the foam designated “Goggle” required the highest gas pressure to maintain a 5 or 10 lpm gas flow at all tested compressions; in other words, it leaked less than the other foams tested. None of the other foams at the highest compressions, 50% or 60%, sealed as well as the goggle foam at 20% compression. Viscoelastic-D was the only other foam tested that sealed at both low and high compressions.

Goggle foam is a non-reticulated polyurethane foam that is latex-free, biocompatible, non-irritating to the skin, resistant to fluid absorption, and compatible with breathable gases. Accordingly, a non-reticulated polyurethane foam is a preferred foam for the cuff 14. Non-reticulated polyurethane foams are available that are soft and comfortable to the user's face. Preferably, the density of the foam is from about 1.4 lb/cu·ft to about 1.8 lb/cu·ft, more preferably, about 1.5 lb/cu·ft. At about 25% compression, the compression load deflection (CLD) of the foam is preferably from about 0.2 psi to about 0.4 psi, more preferably about 0.3 psi. At about 65% compression, the CLD is preferably from about 0.4 psi to about 0.6 psi, more preferably about 0.5 psi. The average pore size of the foam is preferably from about 70 ppi to about 90 ppi, more preferably from about 79 ppi to about 81 ppi, most preferably, about 80 ppi. Particularly preferred foams include ether, non-reticulated polyurethane foams, for example EC80S and EC80F, supplied by Foamex. Another preferred foam is a viscoelastic foam.

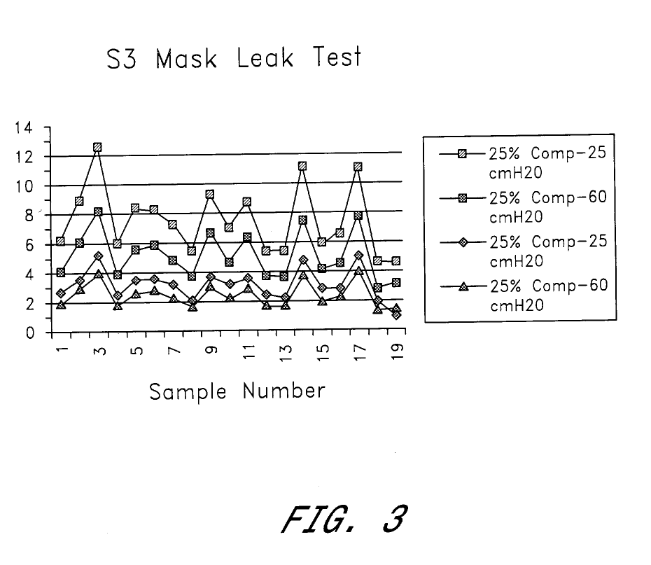

An experiment was performed in which nineteen samples of 0.88 in. high, 80 ppi, non-reticulated polyurethane foam with a firmness of 1.50 were tested for gas leakage. Each sample was tested at both 25% compression and 50% compression at 25 and 60 cm H₂O gas pressure. The results are illustrated in FIG. 3. In general, the leakage at 50% compression was less than half of the leakage at 25% compression at a given pressure.

In certain embodiments, the cuff 14 is adhesively attached to the flange 16. Adhesives suitable for securing the cuff to the dome should be compatible with both the cuff 14 and the flange 16, compatible with the breathable gases, biocompatible, and robust to the environmental conditions under which the face mask will be stored and used. Suitable adhesives are known in the art. Where the dome is made from PVC and the cuff from polyurethane, a preferred adhesive is a UV-curing adhesive. Either or both surfaces of the flange 16 may also be textured to improve adhesion.

With a sealing force of 4 kg (8.8 lb) against a standard resuscitation mannequin face, a preferred embodiment of the disclosed positive pressure full-face mask maintained a positive pressure of about 60 cm H₂O with a leakage of less than about 1.0 L/min. With normal hand pressure against the face, the mask tested to about 60 cm H₂O. This enhanced sealing ability permits single operator bag-valve-mask resuscitation (BVMR), wherein the operator holds the mask against the user's face with one hand and operates the bag with the other. Typically, BVMR requires two operators: one to secure the mask to the user's face and the other to operate the bag.

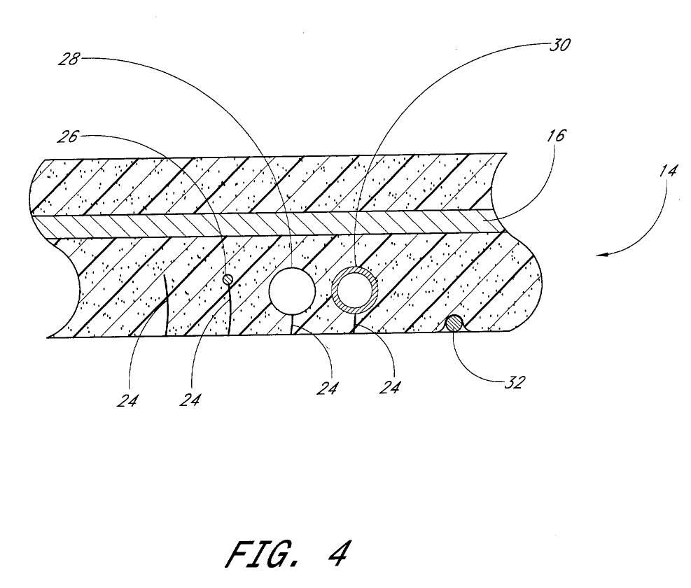

Another advantage of the full-face mask 10 is that the foam cuff 14 seals over facial hair, a shortcoming of other face-mask designs. The mask will even seal over thin cannulae or wires. As illustrated in FIG. 4, larger cannulae 26, for example naso-gastric tubes, may also be used with the disclosed face-mask by simply cutting a slit 24 in the foam cuff 14 and passing the cannula 26 therethrough. The slit illustrated in FIG. 4 is perpendicular to the sealing surface of the cuff; however, the slit may also be angled with respect to the sealing surface. Moreover, the slit may be a simple slit as illustrated, or may be, for example, T-, Y-, J- or cross-shaped, or any other suitable shape. In another preferred embodiment, multiple cannulae 26 may be passed through a single slit in the cuff 14. Alternatively, a slit 24 and a hole 28 may be cut or punched through the foam cuff 14 through which even larger cannulae 30 may be threaded. Also illustrated in FIG. 4 is a cannula 32 passing under the foam cuff 14 without a slit. In any case, the ability of the foam cuff to conform to and mold around the cannula permits the face-mask to provide a good seal to the user's face.

Finally, the cuff 14 of the face-mask is not inflated, and consequently, cannot deflate. Accordingly, the mask may be applied to the patient with no need to inspect the cuff or otherwise prepare it for use.

In another preferred embodiment, the disclosed full-face mask 10 is fabricated in a range of sizes, for example, for infant, pediatric, and adult uses. These sizes may further be subdivided, for example, masks for adult use may further be fabricated in a range of sizes, for example small, medium, and large. Providing a mask sized to fit the user improves both the sealing and comfort of the mask. Preferably, the sizes are color-coded, simplifying selection and reducing confusion in a possibly hectic emergency situation.

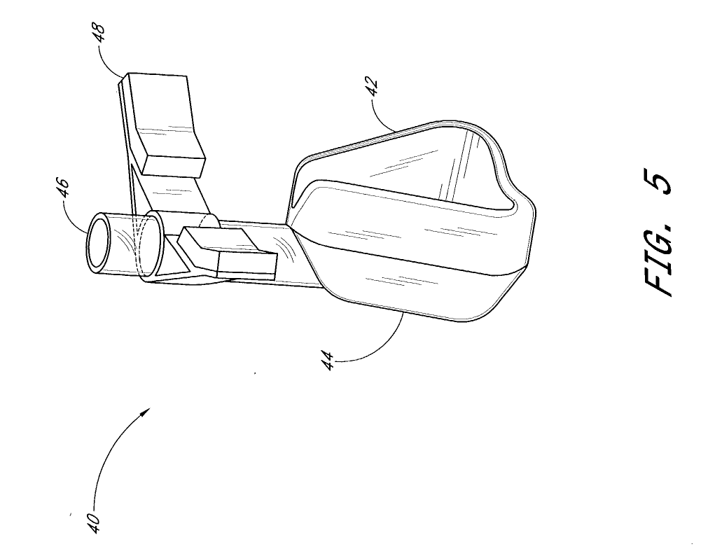

Another type of face mask is a nasal mask. In a nasal mask, the portion corresponding to the dome is referred to as the shell, and the portion corresponding to the cuff is referred to as the cushion. As described above, the cushion on currently available nasal masks are made from silicone or gel-filled silicone. A nasal mask in which the cushion is made from certain types of foam improves user comfort, and hence, compliance with CPAP therapy. A preferred embodiment of a nasal mask is illustrated in FIG. 5. The nasal mask 40 has a foam cushion 42 and a shell 44. The shell may be of any shape or configuration known in the art. For example, the shell may be manufactured in a range of sizes. In another embodiment, the shell is pliable and may be shaped or contoured by the user or a third party to provide an optimal fit. The shell has a gas inlet port 46. In the illustrated embodiment, the gas inlet port 46 is at the top of the shell 44; however, the gas inlet port may also be located at any convenient position on the shell, for example, the bottom of the shell or the portion distal from the user, or at a different angle. The shell of the nasal mask may be fabricated from the same materials as the dome of the full-face mask. Preferably, the shell is PVC. Also provided in the illustrated embodiment is an optional forehead support 48, which helps to stabilizes the nasal mask on the user's face. The nasal mask 40 is held in place by any type of headgear known in the art (not illustrated).

A polyurethane foam is a preferred foam for the cushion 42 of the nasal mask. Preferred polyurethane foams are the same as for the cuff of the full-face mask, described above. A more preferred foam for the cushion 42 is a viscoelastic foam, which is latex-free. As indicated in TABLE I and TABLE II, the viscoelastic foam has some gas permeability, reducing rebreathing. A nasal mask 40 made with a viscoelastic foam cushion reduces skin friction irritation and pressure point ulceration compared to existing masks. Because of the superior seal, gas leakage into the user's eyes is also reduced. The foam is of sufficient thickness to conform to the user's facial features at the interface between the mask and the face, in this case, the region around the nose. The required thickness of the foam will vary with the design of the shell and the particular characteristics of the foam, and is readily determined by those skilled in the art without excessive experimentation.

The embodiments illustrated and described above are provided as examples of certain preferred embodiments of the present invention. Various changes and modifications can be made to the embodiments presented herein by those skilled in the art without departure from the spirit and scope of this invention, the scope of which is limited only by the claims appended hereto.

What is claimed is:

1. A positive pressure full-face mask comprising a dome secured to a foam cuff, wherein the foam cuff contacts a user's face.

2. The positive pressure full-face mask of claim 1, wherein the foam cuff is a non-reticulated polyurethane foam.

3. The positive pressure full-face mask of claim 2, wherein the non-reticulated polyurethane foam is an ether-type non-reticulated polyurethane foam.

4. The positive pressure full-face mask of claim 1, wherein the foam cuff is countered to conform to a human face.

5. The positive pressure full-face mask of claim 1, wherein the dome is polyvinyl chloride.

6. The positive pressure full-face mask of claim 1, wherein the dome is a styrene-butadiene copolymer.

7. The positive pressure full-face mask of claim 1, wherein the dome is flexible.

8. The positive pressure full-face mask of claim 1, wherein the dome further comprises a flange to which the foam cuff is secured.

9. The positive pressure full-face mask of claim 8, wherein the flange is contoured to conform to a human face.

10. The positive pressure full-face mask of claim 8, wherein the cuff is secured to the flange with an adhesive.

11. The positive pressure full-face mask of claim 9, wherein the adhesive is a UV-curing adhesive.

12. The positive pressure full-face mask of claim 1, further comprising a thumb and finger ledge.

13. The positive pressure full-face mask of claim 1, wherein a cannula or wire passes between the foam cuff and the user's face.

14. The positive pressure full-face mask of claim 1, wherein a cannula or wire passes through a slit or hole in the foam cuff.

15. A positive pressure full-face mask comprising a dome secured to a cuff, wherein the mask will hold about 60 cm H₂O or greater gas pressure when held against a user's face with normal hand pressure.

16. The positive pressure full-face mask of claim 15, wherein the cuff is foam.

17. The positive pressure full-face mask of claim 16, wherein the foam cuff is a non-reticulated polyurethane foam.

18. The positive pressure full-face mask of claim 17, wherein the non-reticulated polyurethane foam is an ether-type non-reticulated polyurethane foam.

19. The positive pressure full-face mask of claim 15, wherein the cuff is countered to conform to a human face.

20. The positive pressure full-face mask of claim 15, wherein the dome is polyvinyl chloride.

21. The positive pressure full-face mask of claim 15, wherein the dome is a styrene-butadiene copolymer.

22. The positive pressure full-face mask of claim 15, wherein the dome is flexible.

23. The positive pressure full-face mask of claim 15, wherein the dome further comprises a flange to which the foam cuff is secured.

24. The positive pressure full-face mask of claim 23, wherein the flange is contoured to conform to a human face.

25. The positive pressure full-face mask of claim 23, wherein the cuff is secured to the flange with an adhesive.

26. The positive pressure full-face mask of claim 25, wherein the adhesive is a UV-curing adhesive.

27. The positive pressure full-face mask of claim 15, further comprising a thumb and finger ledge.

28. The positive pressure full-face mask of claim 15, wherein a cannula or wire passes between the foam cuff and the user's face.

29. The positive pressure full-face mask of claim 15, wherein a cannula or wire passes through a slit or hole in the foam cuff.

30. A method of providing a breathable gas to a user comprising
positioning a positive pressure full-face mask comprising a dome secured to a foam cuff over the nose and mouth of the user, wherein the foam cuff contacts the user's face,
applying sufficient pressure to the mask to form a seal between the full-face mask and the user's face, and
providing a breathable gas through an inlet port on the full-face mask.

31. The method of claim 30, wherein the foam cuff is a non-reticulated polyurethane foam.

32. The method of claim 31, wherein the non-reticulated polyurethane foam is an ether-type non-reticulated polyurethane foam.

33. The method of claim 30, wherein the foam cuff is countered to conform to a human face.

34. The method of claim 30, wherein the dome is polyvinyl chloride.

35. The method of claim 30, wherein the dome is a styrene-butadiene copolymer.

36. The method of claim 30, wherein the dome is flexible.

37. The method of claim 30, wherein the dome further comprises a flange to which the foam cuff is secured.

38. The method of claim 37, wherein the flange is contoured to conform to a human face.

39. The method of claim 37, wherein the cuff is secured to the flange with an adhesive.

40. The method of claim 39, wherein the adhesive is a UV-curing adhesive.

41. The method of claim 30, further comprising a thumb and finger ledge.

42. The method of claim 30, wherein a cannula or wire passes between the foam cuff and the user's face.

43. The method of claim 30, wherein a cannula or wire passes through a slit or hole in the foam cuff.

44. A nasal mask comprising a foam cushion.

45. The nasal mask of claim 44, wherein the foam is a viscoelastic foam.

46. The nasal mask of claim 44, wherein the foam is a polyurethane foam.

47. A method of providing CPAP therapy to a user comprising
positioning a nasal mask comprising a foam cushion over the nose of the user,
applying sufficient pressure to the mask to form a seal between the nasal mask and the user's face, and
providing a breathable gas through an inlet port on the nasal mask.

48. The method of claim 46, wherein the foam is a viscoelastic foam.

49. The method of claim 44, wherein the foam is a polyurethane foam.
